(19) United States
(12) Patent Application Publication
(10) Pub. No.: US 2025/0311999 A1
(43) Pub. Date: Oct. 9, 2025

(54) ELECTRONIC STETHOSCOPE

(71) Applicants: Rupak Kumar JHA, Singapore (SG); Meena Kumari SELVARENGARAJU, Singapore (SG)

(72) Inventors: Rupak Kumar JHA, Singapore (SG); Meena Kumari SELVARENGARAJU, Singapore (SG)

(21) Appl. No.: 19/243,027

(22) Filed: Jun. 19, 2025

Related U.S. Application Data
(63) Continuation-in-part of application No. 17/523,397, filed on Nov. 10, 2021.

(51) Int. Cl. A61B 7/04; A61B 5/00; H04R 1/46

(52) U.S. Cl. CPC A61B 7/04; A61B 5/7217; A61B 5/7267; A61B 5/7415; A61B 5/742; H04R 1/46

(57) ABSTRACT

The present invention provides an electronic stethoscope comprising a replaceable chest-piece secured in a fixed housing and covered by a static diaphragm; a rim holding the diaphragm with the chest-piece; an anti-chill ring located at the outer side of the chest piece; a contact sensor mounted on the rim of the chest piece; a main microphone or sound sensor to capture the body sounds; a visible-cue LED indicator to indicate the ON/OFF position of the stethoscope; an ambience microphone configured to capture only the environmental noise and avoids capturing the intended signal component; one or more control buttons for navigating through options shown on a display screen; a remote timer; and a programmable processor to execute machine readable instructions to analyze the signal from the sound sensor comprising the microcontroller, the signal processor for processing the signals and the anti-aliasing filter for anti-aliasing filtering.

### CROSS-REFERENCE TO RELATED APPLICATIONS

This application is a Continuation-in-Part of U.S. patent application Ser. No. 17/523,397, filed on 10 Nov. 2021, which is incorporated herein by reference in its entirety.

### DESCRIPTION OF THE INVENTION

### Technical Field of the Invention

This invention relates to electronic stethoscopes. More specifically, this invention relates to electronic stethoscopes that apply artificial intelligence to compare and analyze readings and which have wireless capabilities allowing remote practice of medicine, or telemedicine.

### BACKGROUND OF THE INVENTION

The stethoscope is a medical device used by doctors, nurses, and other healthcare professionals to listen to sounds from human or animal body. Health care professionals use stethoscopes to listen to the sounds from heart, lungs, arteries and veins, intestines, mother's womb to diagnose based on the sounds received. After well over a century of use, the stethoscope is a ubiquitous diagnostic tool and practitioners have considerable training, experience, and comfort with a stethoscope.

The conventional widely used stethoscope consists of chest piece, flexible rubber tube, and earpiece all connected. Typically, the chest piece itself has two surfaces that may be applied to a patient for auscultation. Theses surfaces are the diaphragm and the bell. The diaphragm is a plastic or fiber glass disc fixed tightly to a circular rim. Behind the diaphragm is a chamber with a conical back opposite the diaphragm to direct sound to an aperture. The bell is a shallow open cup with an aperture in it. The cup shape of the bell directs sounds to the aperture. The apertures from the diaphragm and the bell lead to passages that lead to the flexible rubber tube. The flexible rubber tube in turn conducts sound to the earpiece(s). The bell transmits low frequency sounds, whereas the diaphragm transmits high frequency sounds. When the diaphragm of a stethoscope is placed on the human or animal body, the diaphragm vibrates according to body sounds, creating acoustics pressure waves which are directed to the aperture and travel up the tube to the earpiece. Health care professionals place the earpiece on their ears to listen to the sound from diaphragm.

With constantly improving electronics and communication technology, the field of telemedicine has consistently expanded in its reach and in its fields of application. With mobile communication technology, less hard infrastructure is needed for communicating over great distances, so that patients in remote locations may still have access to practitioners. With the miniaturization of electronics and the improvement of data transmission, digitized information can be gathered in remote locations and transmitted for analysis or stored. All of this was expanding the reach of telemedicine. Additionally, while it is natural to think of the patient as a person, the patient could be an animal. Telemedicine has extended the reach of species specialists, and as a result, telemedicine has expanded among veterinarians as well. Therefore, when the terms patient or body are used, the reference may also be to an animal.

In 2019-2020, the globe was hit by a pandemic commonly called COVID-19, also known as Coronavirus pandemic. This pandemic was caused by severe acute respiratory syndrome coronavirus 2 (SARS-COV-2). Virus spreads though air when infected person nearby cough, breathe, or sneeze. It may also spread when contaminated surfaces are touched. In general, travel and personal interaction were greatly reduced during the pandemic. Additionally, due to concern about disease spread by physical contact, doctors and nurses faced difficulty carrying the stethoscope around, sterilizing it, and using it with the Personal Protection Equipment (PPE) kit worn by them. Due to the pandemic, there was a continued and expanding need for remote doctor consultation (Tele-consultation), which spurred the application of telemedicine even further. Tele-consultation and telemedicine still require the ability to collect diagnostic information such as provided by a traditional stethoscope.

Although electronic stethoscopes provide advantages, there are problems associated with them as well. The heighted sensitivity of electronic stethoscopes can introduce noise problems which must be addressed if the electronic stethoscope is to be effective. In many environments, ambient noises are significant. Another source of noise is located at the stethoscope itself. If the diaphragm face of the stethoscope is moving with respect to the surface it is contacting, clothing or skin, a significant noise component is generated. Both of these noise components should be addressed to gain the benefits of an electronic stethoscope.

When an electronic stethoscope is used for remote medical consultations, the user of the electronic stethoscope may not be a practitioner. To ensure acquisition of good signals and measurements, it is desirable that a remote practitioner has some control over the acquisition of a measurement. One factor associated with this is the duration of the measurement wherein a minimal length of time for a measurement may establish a higher quality measure and provide better diagnostic information. Additionally, diagnoses often entail comparison to baselines. Information associated with a given patient captured along with the measurement provides a better selection of the baseline.

A patent application US2009211838A1 entitled “Floating Ballast Mass Active Stethoscope or Sound Pickup Device” discloses an active stethoscope or other sound detection device, including a diaphragm, at least one floating mass mounted to the diaphragm (at atleast one coupling point of the diaphragm), and an acoustic transducer mounted to the floating mass. Preferably, each floating mass is configured and mounted so that as each floating mass and each coupling point of the diaphragm move in sympathy with acoustic waves (to be detected) that impinge on the diaphragm, the acoustic transducer rides with and is stabilized by the floating mass to which it is mounted and the diaphragm is stabilized by each floating mass. The acoustic transducer can be of any of many different types. For example, it can be a microphone, or an optical, capacitive, or inductive transducer. The diaphragm can have an isolating portion which absorbs acoustic surface wave energy incident thereon, or otherwise prevents or reduces transmission of acoustic surface waves through the isolating portion between regions of the diaphragm.

Another patent application No. US2016100817A1 entitled “Systems, devices, and methods for capturing and outputting data regarding a bodily characteristic” discloses systems, devices, and methods are provided for capturing and outputting data regarding a bodily characteristic, wherein in one embodiment, a hardware device can operate as a stethoscope with sensors to detect bodily characteristics such as heart sounds, lung sounds, abdominal sounds, and other bodily sounds and other characteristics such as temperature and ultrasound. The stethoscope can be configured to work independently with built solid-state memory or SIM card. The stethoscope can be configured to pair via a wireless communication protocol with one or more electronic devices, and upon pairing with the electronic device(s), can be registered in a network resident in the cloud and can thereby create a network of users of like stethoscopes.

Another patent application No. US2017340306A1 entitled “Abdominal statistics physiological monitoring system and methods” discloses an abdominal statistics system including a low profile rapidly deployable sensor element having an acoustic sensor and vibration actuator that can be conveniently attached to the abdomen of a patient. The system acquires acoustic signals as gastrointestinal (GI) sounds, processes these signals, and provides actionable data to patients and their providers.

A patent application No. US2021244379A1 entitled “Stethoscope and electronic auscultation apparatus” discloses a stethoscope that includes a support base; and a detection unit that is supported by the support base and detects a sound generated from an object to be measured, in which the detection unit has a piezoelectric film disposed to face the support base in at least a portion for detecting the sound generated from the object to be measured and convexly curved to a side opposite to the support base, the piezoelectric film includes a piezoelectric layer having two main surfaces facing each other, a first electrode provided on a main surface on a support base side of the two main surfaces, and a second electrode provided on a main surface on a side opposite to the support base, and a strain generated in the piezoelectric film due to the sound generated from the object to be measured is detected as a vibration signal.

However, the prior art exhibits several drawbacks and limitations that remain unresolved. Accordingly, there is a need for an improved electronic stethoscope that overcomes the shortcomings.

### SUMMARY OF THE INVENTION

The present invention discloses overcomes the drawbacks of the existing electronic stethoscopes to provide an improved hand-held electronic stethoscope comprising a fixed housing, a housing with a static diaphragm at a surface of the housing and electronics within the housing wherein the electronics of the electronic stethoscope further comprises: a sound sensor; a programmable processor chip; a battery; a contact sensor; a display screen; user interface controls; a port; wireless communication elements; and other electronic elements. The electronics are distributed variously about the electronic stethoscope on circuit boards, etc. with some visible and accessible to users. The electronic stethoscope further comprises a chest piece secured in the fixed housing, wherein the chest piece is replaceable in nature, a chest piece holder to hold the chest piece in place, an ambience microphone, a sound sensor, an audio tube, an anti-chill ring, a visible-cue LED indicator, a contact sensor, and a programmable processor.

The diaphragm is located at a surface of the fixed housing where the diaphragm is static. The diaphragm further comprises a sensor module facilitating detection of a body sound. The electronic stethoscope further comprises a chest piece located behind the diaphragm and secured in the fixed housing, a chest-piece holder, where the chest piece has an aperture through it. The chest piece is positioned within the fixed housing behind the diaphragm, and the sound sensor is positioned to receive sound from the aperture in the chest piece and record the sound transmitted by the diaphragm and chest piece. The sound sensor comprises a transducer and processor for filtering, conditioning, and converting from analog to digital signals. The sound sensor is located outside of and behind the chest piece within the fixed housing. Further, the aperture in the chest piece facilitates placement of the sound sensor at the end of the audio tube. The ambience microphone facilitates recording of the ambient sounds, wherein the ambience microphone is located internally in the fixed housing of the electronic stethoscope.

Further, the anti-chill ring is located at the outer side of the chest piece, facilitating prevention of the direct contact of the chest piece with the subject's body. Further, the visible-cue LED indicator to display the ON/OFF position of the electronic stethoscope, wherein the visible-cue LED indicator provides visual indication to the user that the electronic stethoscope is ready to capture the audio, and upon capturing the audio, the visible-cue LED indicator provides indication to the user that the electronic stethoscope may be removed from position indicating that the measurement has been captured successfully. The contact sensor is located on the rim, wherein the contact sensor senses the force of contact and facilitates determination of the establishment of stable contact of the electronic stethoscope with a body. Additionally, the programmable processor executes the machine-readable instructions, wherein, the programmable processor monitors the contact sensor for a contact signal indicating that the diaphragm is in position and the programmable processor operates a remote timer to control the duration of a recording from the sound sensor.

The sound sensor transmits a signal to the programmable chip for storage, analysis, additional processing, transmission to other elements. In some embodiments, programmable chip itself has wireless communication capabilities and can transmit the signal received from the sound sensor. The programmable chip executes machine readable instructions to analyze the signal from the sound sensor, drive the display, receive signals from a user interface, receive signals from the contact sensor, and in general operate and coordinate the other elements of the electronic stethoscope. The machine-readable instructions for the programmable chip may modified via wireless communications or the port which also provides a means for recharging the battery.

The contact sensor is located proximal to the rim. The contact sensor detects when the rim is in contact with the body and sends a signal to the programmable chip. With confirmation of contact between the diaphragm and the body, the programmable chip initiates a remote timer while receiving and recording signals from the sound sensor. The duration of the remote timer may be adjusted by the user on location or the duration may be remotely adjusted by a consultant. The electronic stethoscope may provide cues such as audible cues to indicate when a timer has been initiated, and when the electronic stethoscope may be moved. The electronic stethoscope produces audible cues while it is in operation, wherein the audible cues indicate the beginning of recording, ending of recordings, error conditions, etc. In this way, it is assured that a sound sample is of sufficient length for diagnostic purposes. Additionally, the electronic stethoscope may provide visible cues with the help of visible-cue LED indicator, providing indications to the user when the electronic stethoscope is ready to capture the audio and when the audio capturing is ceased.

In some embodiments of the electronic stethoscope, the signal from the contact sensor may prompt other steps by the programmable chip. Motion of the diaphragm along a surface such as skin or clothing can generate a surge of noise. For contact sensors capable of measuring force, a minimum threshold of force is interpreted by the programmable chip as indicating that the electronic stethoscope is firmly in place and stationery. With the stethoscope in place, signals from the sound sensor can be recorded and processed without a surge of noise into the signal and resulting audio file.

### BRIEF DESCRIPTION OF THE DRAWINGS

Additional utility and features of the invention will become more fully apparent to those skilled in the art by reference to the following drawings, which illustrate some of the primary features of preferred embodiments.

FIG. 1 is a side perspective view of an embodiment of an electronic stethoscope.

FIG. 2 is a perspective view of the listening end of an embodiment of an electronic stethoscope.

FIG. 3 is a perspective view of an embodiment of a chest piece in an embodiment of an electronic stethoscope.

FIG. 4 is an exploded view of an embodiment of the electronic stethoscope.

FIG. 5 is a cross-sectional view of an embodiment of the electronic stethoscope.

FIG. 6A is a top view of the central circuit board, according to an embodiment of the invention.

FIG. 6B is a bottom view of the central circuit board, according to an embodiment of the invention.

FIG. 7A is a top view of the sound sensor, according to an embodiment of the invention.

FIG. 7B is a bottom view of the sound sensor, according to an embodiment of the invention.

FIG. 8A is a top view of the ambience microphone, according to an embodiment of the invention.

FIG. 8B is a top view of the ambience microphone, according to an embodiment of the invention.

FIG. 9 is a perspective view of a tribosensor facilitating detection of ultra-sensitive audio, according to an embodiment of the invention.

FIG. 10 illustrates a flowchart disclosing the working mechanism of the pressure signal to capture the audio recording.

FIG. 11 illustrates a flowchart disclosing the working mechanism of the remote timer.

FIG. 12 and FIG. 13 illustrates the flowchart disclosing the process of building a random forest built with a bootstrapped data set, according to an embodiment of the invention.

FIG. 14 illustrates the flowchart disclosing the diagnostic inference using the random forest built with the bootstrapped data set, according to an embodiment of the invention.

FIG. 15 is a perspective view of an acoustic electronic stethoscope disclosing the electrodes for the measurement of heart's electrical activity, according to an embodiment of the invention.

### DETAILED DESCRIPTION OF EMBODIMENTS

The terms “electronic stethoscope” and “stethoscope” are also used interchangeably and refer to a stethoscope incorporating electronic components for signal processing, amplification, or transmission, unless otherwise specified.

As used herein, the terms “chest piece” and “replaceable chest piece” are used interchangeably and shall be understood to refer to the same component or structure.

Furthermore, the terms “static diaphragm” and “diaphragm” are used synonymously throughout this disclosure to refer to a vibratory surface or membrane used in acoustic or electronic stethoscope applications.

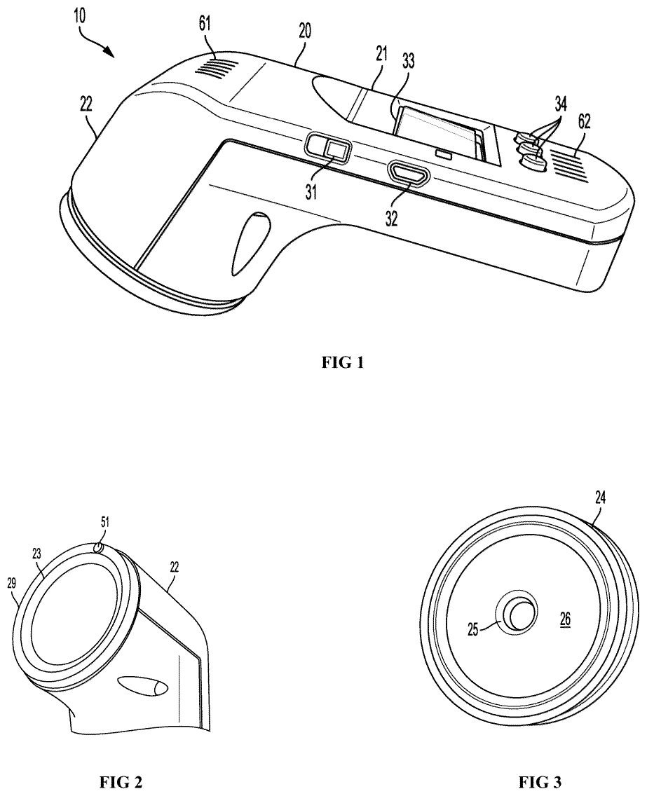

FIG. 1 is a side perspective view of an embodiment of an electronic stethoscope 10, wherein the electronic stethoscope 10 comprises a fixed housing 20 that houses electronics of the electronic stethoscope 10 and has multiple apertures for user controls, measurements, and communications. The electronic stethoscope 10 comprises a power switch 31 on a handle 21, to provide manual control of the state of electronic stethoscope 10. Further, a port 32 located on the handle 21 of the electronic stethoscope 10 provides a connection for charging the electronic stethoscope 10. The configuration of the port 32 is not limited to that shown in FIG. 1. Any desirable port may be employed. The port 32 also provides another means of input and output for electronic stethoscope 10. Information may be downloaded through the port 32, and the port 32 may also be used to upload updates of information and firmware to the electronic stethoscope 10.

The electronic stethoscope 10 further comprises a display screen 33 and control buttons 34 on the handle 21 of the electronic stethoscope 10, that provides a user interface with the electronic stethoscope 10. The display screen 33 may be an LED display or any suitable display. In one embodiment of an electronic stethoscope 10, the control buttons 34 comprises five buttons for navigating through options shown on display screen 33 for manual control of the electronic stethoscope 10. Any suitable interface may be used for the control buttons 34. In some embodiments of the electronic stethoscope 10, the control buttons 34 may be micro-switch push buttons or any similar suitable push button. Other embodiments of electronic stethoscope 10 may employ capacitive touch sensor buttons to navigate through options on display screen 33. Still other embodiments may employ touch sensitive screens. Control buttons 34 may comprise a button each for “Up”, “Down”, “Record”, “Mode” and “Select”. These allow a user to move through options displayed on display screen 33 and select choices in decision trees or select functions to operate electronic stethoscope 10. Additionally, the number of input buttons may be changed as desired. In one embodiment of an electronic stethoscope 10, the display screen 33 such as the LED display is touch-sensitive, that provides the control button 34 options on the display screen 33, by touch, for navigating through options.

A head 22 on the electronic stethoscope 10 houses some of the audio components of the electronic stethoscope 10. In some embodiments of electronic stethoscope 10, the head 22 may have ambient apertures 61 to allow passage of sound between the interior and exterior of the fixed housing 20. In some embodiments of the electronic stethoscope 10, the handle 21 may have sound apertures 62 to allow passage of sound between the interior and exterior of the fixed housing 20.

FIG. 2 is a perspective view of the head 22 of an embodiment of electronic stethoscope 10. A diaphragm 23, acting as a surface of the fixed housing 20, covers the end of the head 22, and helps to “pick up” or capture higher and lower frequency body sounds when the head 22 of the electronic stethoscope 10 is applied to the body. The head 22 contains a chest piece, wherein the chest piece is positioned behind the diaphragm 23 to direct and transmit sounds received from the diaphragm 23. A rim 29 holds the diaphragm 23 in place.

FIG. 3 illustrates a perspective view of an exemplary embodiment of a chest piece 24 configured to direct acoustic signals received from the diaphragm 23. The chest piece 24 is removably attachable to facilitate enhanced versatility, hygiene, and maintenance. The detachable configuration of chest piece 24 permits removal and replacement without the use of specialized tools. The modular nature of chest piece 24 enables various operational scenarios, including, but not limited to: hygienic replacement between patient uses to reduce the risk of cross-contamination; adaptability for use across different patient types, including adult, pediatric, and veterinary subjects, in accordance with varying auscultation requirements; and simplified substitution in the event of wear or physical damage, thereby extending the operational lifespan of the electronic stethoscope 10 without necessitating replacement of the entire device. In certain embodiments, the chest piece 24 is fabricated from a metallic material, such as aluminum or zinc alloy. An aperture 25 is formed through the chest piece 24 to permit transmission of body sounds therethrough.

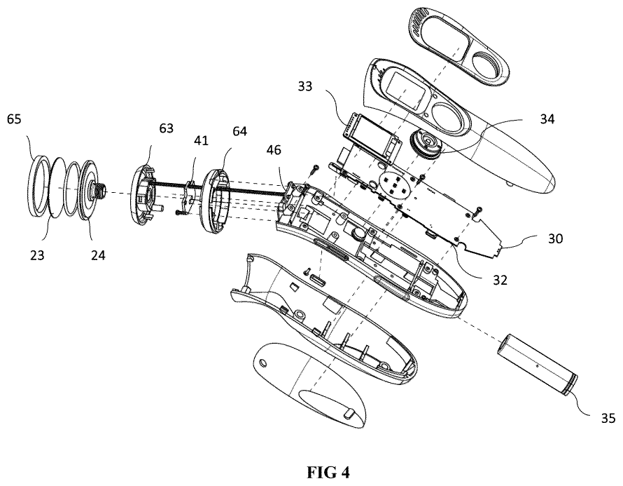

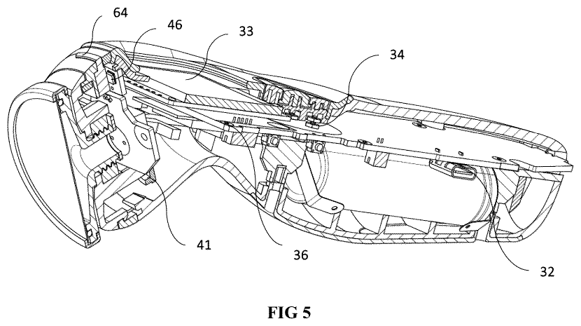

FIG. 4 is an exploded view of an embodiment of the electronic stethoscope 10. In the embodiment shown in FIG. 4, the electronic stethoscope 10 discloses an anti-chill ring 65, the diaphragm 23, the chest piece 24, a chest-piece holder 63, a main microphone or sound sensor 41, a visible-cue LED indicator 64, an ambience microphone 46, an LED display 33, one or more control buttons 34, a replaceable battery 35, and a central circuit board 30. The anti-chill ring 65 located at the outer side of the chest piece 24 facilitates prevention of the direct contact of the chest piece with the body. The anti-chill ring 65 is especially important in cold environments or when examining infants, children, or anxious patients, as cold can cause muscle tensing or sudden shivering, and will interfere with the accurate auscultation. Further, the anti-chill ring 65 forms a barrier between the stethoscope 10 and the body that reduces skin contact, and helps to minimize the contamination, as it is easier to disinfect. Additionally, the anti-chill ring 65 creates a seal between the patient's skin and the diaphragm 23, thus improving the sound transmission, according to an embodiment of the invention.

The head 22 of the electronic stethoscope 10 houses the chest piece 24, wherein the chest piece 24 is covered by a diaphragm 23. A rim 29 is provided to secure the diaphragm 23 to the chest piece 24, thereby maintaining proper alignment and acoustic coupling. A contact sensor 51 is mounted on or integrated with the rim 29 of the chest piece 24 and is configured to directly detect pressure exerted by the subject's body during auscultation. The contact sensor 51 ensures stable contact between the chest piece 24 and the subject's body and is operable to trigger audio attenuation during periods of unstable or insufficient contact. In one embodiment, the contact sensor 51 is further configured to measure the pressure exerted at the interface between the diaphragm 23 of the electronic stethoscope 10 and the subject. When the pressure falls below a predefined threshold, the stethoscope 10 attenuates the audio signal to suppress the ambience noise. Conversely, when the pressure exceeds the threshold value, the audio signal is processed without attenuation, ensuring optimal signal fidelity. The aperture 25 allows for the positioning of the sound sensor 41 either directly behind the diaphragm 23 or within an associated audio tube. The chest piece 24 is further secured within the fixed housing 20, ensuring mechanical stability and acoustic isolation during operation.

In certain embodiments, the chest-piece holder 63 is provided and configured to securely retain a replaceable chest piece 24 of the electronic stethoscope 10. The chest-piece holder 63 comprises a connection mechanism configured to enable secure yet releasable attachment of the chest piece 24, thereby facilitating quick release while ensuring that the chest piece 24 remains protected, undamaged, and readily accessible when not in use. The chest-piece holder 63 is contoured to substantially conform to the external geometry of the chest piece 24, thereby providing a snug and stable fit that prevents inadvertent dislodgement. Furthermore, the chest-piece holder 63 is operable to shield the sound sensor 41 that is operatively associated with the chest piece 24, from dust, mechanical impact, and environmental contaminants, thereby contributing to the maintenance of the performance accuracy and operational longevity of the electronic stethoscope 10.

The electronic stethoscope 10 further comprises a primary microphone or sound sensor 41 and an ambience microphone 46, wherein the orientation of the ambience microphone 46 differs from that of the sound sensor 41. As a result of the differing orientation, the ambience microphone 46 is configured to selectively capture environmental or ambient noise while substantially excluding the intended auscultatory signal. In certain embodiments, the sound sensor 41 is positioned externally relative to and rearward of the chest piece 24. In some implementations of the electronic stethoscope 10, the ambience microphone 46 is embedded within the fixed housing 20 of the electronic stethoscope 10 and is operable to directly sample ambient noise from the environment.

The sound sensor 41 of the electronic stethoscope 10 is configured to transduce acoustic signals transmitted through the diaphragm 23 and directed by the chest piece 24 into corresponding electrical signals, wherein the signals are then transmitted to one or more electronic components of the electronic stethoscope 10 for further processing. According to one embodiment, the chest piece 24 is securely mounted within the fixed housing 20, and the sound sensor 41 is positioned externally relative to and behind the chest piece 24, thereby optimizing acoustic coupling and sensor protection. In certain embodiments, the sound sensor 41 comprises a transducer configured to detect the acoustic signals, a signal conditioner for preprocessing the analog signals, an analog-to-digital (AD) converter for digitizing the conditioned signals, a signal filter for noise reduction and bandwidth control, and an interface circuit for communicating the processed signals to subsequent processing units within the electronic stethoscope 10.

In one embodiment, the ambience microphone 46 facilitates the implementation of adaptive or smart noise-level settings, thereby enabling the electronic stethoscope 10 to dynamically compensate for variations in environmental noise, facilitating enhancement of the quality and clarity of the intended signal acquired by the sound sensor 41. The ambience microphone 46 generates a signal corresponding to the detected ambient sounds, which is subsequently subjected to phase inversion and signal processing. The inverted signal is then combined with the signal acquired by the sound sensor 41 to perform noise cancellation, thereby mitigating the influence of ambient noise on the auscultatory signal captured by the electronic stethoscope 10.

The electronic stethoscope 10 further comprises a central circuit board 30 configured to support various signal acquisition, processing, and storage functionalities. In certain embodiments, the central circuit board 30 includes a signal acquisition and processing module comprising a microcontroller, a signal processor configured to process auscultatory and ambient signals, and an anti-aliasing filter configured to perform anti-aliasing filtering on the acquired signals prior to digital conversion or processing. The electronic stethoscope 10 further includes a port 66 operable to receive a portable memory card for the purpose of storing processed or raw data corresponding to the detected signals. Additionally, a charging port is provided on the electronic stethoscope 10, configured to enable power supply and charging of the internal power source or battery of the device.

Moreover, in some applications, it is desirable to control the duration of the measurement recorded by electronic stethoscope 10. The measurement commences when the contact sensor 51 indicates that the diaphragm 23 is in contact with the body and the diaphragm 23 is in position. If the electronic stethoscope 10 is left in contact longer than needed, the remote timer in the electronic stethoscope 10 terminates the audio recording and captures the audio only till the predetermined duration set by the remote timer. Additionally, recording the audio using the remote timer allows the remote user to control the time of measurement. According to an embodiment of the invention, the remote timer or the remote adjustments allow to change settings of the electronic stethoscope 10 remotely. The duration of the measurement can be set remotely.

The electronic stethoscope 10 comprises various electronic components, including a programmable chip 36 configured and programmed to perform the real-time signal processing operations, wherein the operations may include, but are not limited to, signal processing, encapsulation, analysis, comparison, and wireless transmission of the acquired data to external devices or networked systems. The programmable chip 36 operates in conjunction with firmware that is updatable and supports multiple wireless communication protocols, including Bluetooth, Wi-Fi, 4G, and 5G.

In certain embodiments, the electronic stethoscope 10 is configured to communicate wirelessly with external servers or cloud-based platforms directly via Bluetooth and/or Wi-Fi, without requiring an intermediary computing or communication device. In one embodiment, the Bluetooth module of the electronic stethoscope 10 is configured to remain continuously active without requiring user-initiated activation. The continuously active functionality of the Bluetooth module facilitates improved workflow efficiency, particularly in clinical environments where time sensitivity and ease of use are essential. Furthermore, the design of the electronic stethoscope 10 eliminates the need for manual activation, thereby reducing the potential for missed or failed connections during patient examinations, thus enhancing the overall usability and reliability in healthcare settings, contributing to improved clinical outcomes and workflow productivity.

The electronic stethoscope 10 discloses a battery 35 mounted on the bottom side of central circuit board 30, wherein the battery 35 is rechargeable and is electrically coupled to a charging port 32 configured to receive power from an external power source. In certain embodiments, the electronic stethoscope 10 is further configured to support wireless signal transmission and reception in at least Bluetooth and Wi-Fi communication modes. The wireless communications may include encrypted data formats to ensure secure transmission of the data.

According to certain embodiments of the invention, the electronic stethoscope 10 includes a provision for retaining the diaphragm 23 in a fixed or static position. In such embodiments, the electronic stethoscope 10 is configured to utilize one or more sensors to measure mechanical vibrations required for auscultation, while avoiding reliance on air-coupled transmission within the interior of the chest piece 24, enhancing the signal fidelity and reducing the susceptibility to airborne noise and pressure fluctuations. The implementation of a static diaphragm 23 further enables the use of a broader range of materials in its construction, thereby providing increased flexibility in the design and optimization of acoustic and mechanical properties. In one embodiment, the static diaphragm 23 incorporates a sensor module configured to detect acoustic signals or body sounds emanating from the subject. The sensor module may be embodied as a directional sensor module, including, for example, a one directional sensor or a unidirectional sensor that improves noise cancellation performance and enhances the clarity and accuracy of body sound acquisition. Additionally, the integration of the sensor module within the electronic stethoscope 10 may obviate the need for a separate microelectromechanical system (MEMS) transducer, thereby simplifying the overall device architecture and potentially reducing manufacturing complexity and cost.

According to an embodiment of the invention, noise cancellation is based on the vibrational sensor, wherein the electronic stethoscope 10 makes use of the unidirectional vibrational sensor instead of omnidirectional microphone, that filters the ambient noise coming from other directions. The one-directional vibrational sensor is used as an alternative to a traditional omnidirectional microphone, facilitating capturing of the body sounds. The omnidirectional microphones are designed to capture sound equally from all directions, and are effective in quiet environments, however, the omnidirectional microphone are highly susceptible to ambient noise, including conversations or movements in nearby areas, equipment noise in clinical settings, environmental sounds in home or outdoor monitoring conditions etc., that compromises the signal-to-noise ratio (SNR), making it difficult to extract clean and diagnostically useful auscultation signals.

According to an embodiment of the invention, the unidirectional vibrational sensor is configured to detect the mechanical vibrations traveling through the body or the surface on which it is placed, by avoiding the capture of ambient noise from all the directions. Further, the unidirectional vibrational sensor facilitates targeted signal capture, as the sensor primarily detects vibrations perpendicular to the contact surface, i.e., the chest wall or skin, where heart and lung sounds are the strongest, ensuring the capture of the audio signals by ignoring the ambient sounds. Moreover, as the unidirectional vibrational sensor displays low sensitivity to acoustic noise, such as room conversations or background equipment sounds, resulting in natural noise cancellation, without the need for extensive digital post-processing. Further, the unidirectional vibrational sensor can operate at lower power levels compared to the high-performance omnidirectional microphones, thus making them ideal for portable or battery-powered electronic stethoscope 10.

Additionally, by replacing the omnidirectional microphone with the unidirectional vibration sensor allows the system to focus on the diagnostically relevant body vibrations while rejecting the ambient noise, thus significantly enhancing the reliability and performance of the electronic stethoscope 10 in real-time. The reduced noise settings and better directional sensitivity of the electronic stethoscope 10 leads to significantly clear and highly accurate recordings of physiological sounds.

Further, the electronic stethoscope 10 displays the capability to set the noise-cancellation level based on the environmental noise in which the electronic stethoscope 10 is being used, such as in a loud setting or a quiet setting. The electronic stethoscope 10 can be equipped with smart ambient noise detection, wherein the noise level is pre-set in the electronic stethoscope 10 where the noise levels such as the quiet setting (<40 dB ambient noise), and the loud setting (>60 dB ambient noise) are defined. In one of the embodiments of the invention, a smart ambient noise detection feature is using adaptive filters that adjust their coefficients automatically to adapt to changing signal characteristics of the ambient noise. The electronic stethoscope 10 detects the noise in the captured audio through the ambience microphone 46 and categorizes into quiet setting such as quiet room or a clinic, with minimal background interference; and loud setting such as emergency room, intensive care unit with multiple machines, sirens, alarms, etc., wherein the electronic stethoscope 10 automatically selects the noise level based on ambient noise captured by the ambient microphone 46 in the audio recording. Additionally, due to its strategic placement, the ambient microphone 46 is configured to capture only the ambient noise. A noise profile is generated based on the captured ambient noise, and a corresponding noise level is determined. The electronic stethoscope 10 then utilizes the determined noise level as part of an intelligent noise-adjustment mechanism, which is applied to the audio signal received from the sound sensor 41, wherein the audio signal comprises both the intended signal and the ambient noise component.

FIG. 5 is a cross-sectional view of an embodiment of the electronic stethoscope 10. In the embodiment shown in FIG. 5, the electronic stethoscope 10 discloses the visible-cue LED indicator 64, the ambience microphone 46, the main microphone or the sound sensor 41. The control buttons 34 on electronic stethoscope 10 are configured to allow a user to select settings for the operation of electronic microscope 10 via inputs into microcontroller, or programmable chip, 36. The electronic stethoscope 10 further comprises a port 32 configured to enable connection with an external power source for charging the internal battery. According to an embodiment of the invention, the electronic stethoscope 10 includes a visible-cue LED indicator 64 configured to provide visual feedback to the user during self-examination. When the subject applies the electronic stethoscope 10 to their body, the visible-cue LED indicator 64 illuminates in green, indicating that the stethoscope 10 is powered ON and ready to take the reading, and is in an active state for a predetermined duration, such as 30 seconds, during which audio data acquisition is performed. Upon completion of the data acquisition period, the visible-cue LED indicator 64 changes to for example: red, thereby signaling to the subject that the measurement has been successfully completed and that the electronic stethoscope 10 may be safely removed. In some embodiments, the duration of the active acquisition period may be predefined or dynamically adjustable. The adjustment of the duration may be performed locally by the user through an interface, or remotely by a healthcare professional via a network connection, thus enabling customization of the measurement protocol according to clinical needs.

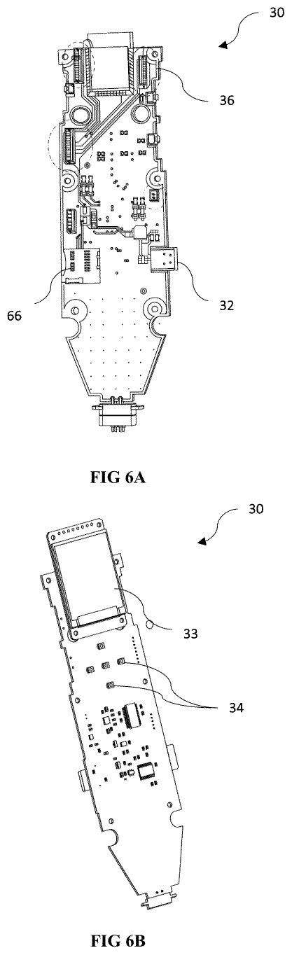

FIG. 6A is a top view of the central circuit board 30, according to an embodiment of the invention, the central circuit board 30 comprising the port 32 configured to charge the battery 35 of the electronic stethoscope 10, a port 66 configured to insert a portable memory card used to store the data, and the programmable chip 36 or the signal acquisition and processing board comprising the microcontroller, the signal processor configured to process the signals and the anti-aliasing filter configured to perform anti-aliasing filtering. The programmable chip 36 executes machine instructions to interact with and control other elements of electronic stethoscope 10 and apply algorithms to process and analyze the signal received from sensor chip 43.

FIG. 6B illustrates a bottom view of the central circuit board 30, in accordance with an embodiment of the invention. As shown, the central circuit board 30 includes the LED display screen 33 configured to provide visual feedback or display information related to device status, settings, or measurement results. Additionally, the central circuit board 30 comprises ports for the control buttons 34, operable to receive the user input for controlling various functions of the electronic stethoscope 10, such as power on/off, audio gain adjustment, mode selection, or wireless connectivity options. The arrangement of the display screen 33 and the ports of the control buttons 34 on the bottom side of the circuit board 30 facilitates ergonomic integration within the fixed housing 20 of the electronic stethoscope 10.

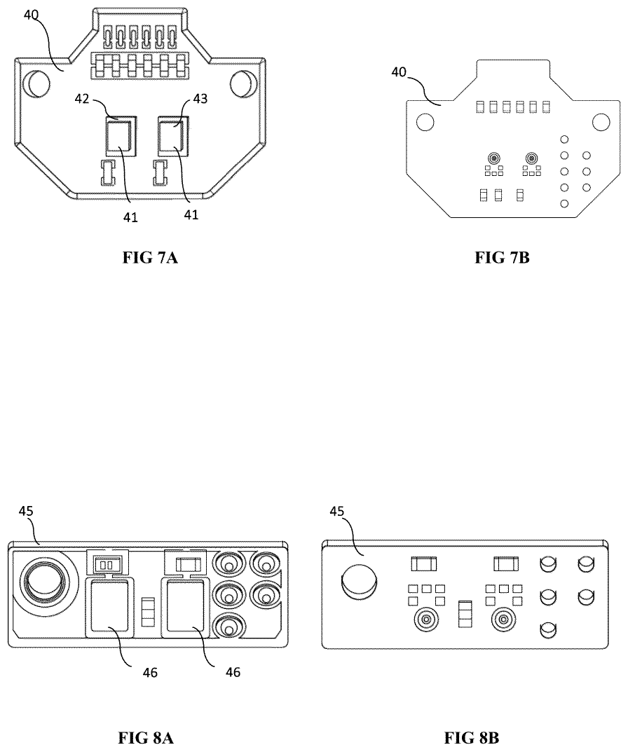

FIG. 7A and FIG. 7B is a top view and the bottom view of the sound sensor 41, according to an embodiment of the invention. In the embodiment shown in FIGS. 7A and 7B, the electronic stethoscope 10 further comprises a primary microphone or the sound sensor 41, facilitating capturing of the sound waves. According to the invention, the orientation of the ambience microphone 46 is different than the main microphone or the sound sensor 41, due to which the ambience microphone 46 is configured to capture only the environmental noise and avoids capturing the intended signal component. The sound sensor 41 comprises multiple components, sound transducer 42 and sensor chip 43. The sound transducer 42 records the sound waves, converts them to an electrical signal, and transmits the resultant signals to sensor chip 43 of sound sensor 41, which performs signal conditioning, conversion of the signal from analog to digital, anti-aliasing filtering, and feeds the signal to an 12S interface for communication to a central processor for analysis, external transmission, etc. The sound transducer 42 may be a MEMS (microelectromechanical system) sensor, or any other suitable sound transducer, and may itself have filtering and conditioning capabilities. The sound sensor 41 located on the first peripheral circuit board 40 is situated to record sounds conducted to it through the audio tube 27. In some embodiments of electronic stethoscope 10, a second sound sensor, or microphone is used to directly sample ambient noise. Furthermore, upon detecting contact between the electronic stethoscope 10 and the subject's body, the electronic stethoscope 10 initiates audio signal capture immediately, based on the signal from the contact sensor 51 indicating that the stethoscope 10 is static, without a delay for additional audio detection.

FIG. 8A and FIG. 8B is a top view and bottom view of the ambience microphone 46, according to an embodiment of the invention. In the embodiment illustrated in FIG. 8A, the electronic stethoscope 10 comprises an ambience microphone 46 configured to detect and record ambient environmental sounds. The signal generated by the ambience microphone 46, corresponding to the recorded ambient noise, is subjected to phase inversion and is subsequently processed in combination with the signal received from the primary sound sensor 41. The processing operation is performed to attenuate or cancel the ambient noise component present in the signal captured by the sound sensor 41, thereby isolating and preserving the intended diagnostic signal corresponding to body sounds. The processed signal is transmitted to a central processor for further analysis or storage.

The ambience microphone 46 is positioned at the rear of the head 22 of the electronic stethoscope 10 and is oriented such that it is directed toward the ambient environment to optimize its ability to capture ambient noise. In some embodiments, the ambience microphone 46 detects the ambient sound transmitted through the fixed housing 20. In other embodiments, as shown in FIG. 1, the ambience microphone 46 is directly exposed to the external environment via the apertures 61, thereby enabling efficient capture of surrounding noise. According to certain embodiments, the ambience microphone 46 is specifically configured to detect and record ambient noise exclusively, without capturing or responding to the diagnostic body sounds. As such, the ambient noise signal generated by the ambient microphone 46 is inverted and utilized in a noise cancellation algorithm that operates on the composite signal comprising both ambient noise and the intended body sounds captured by the sound transducer 42. The obtained output is a noise-reduced signal that substantially represents only the intended body sounds detected by the sound transducer 42, thereby improving the clarity and diagnostic value of the auscultated intended signal.

Moreover, the ambience microphone 46 is mounted on a second peripheral circuit board 45 along with any peripheral chips for signal conditioning and processing. The ambience microphone 46 may itself be a MEMS (microelectromechanical system) sensor or any other suitable sound transducer, and may itself have filtering and conditioning capabilities.

Some embodiments of the invention disclose a contact sensor 51 located proximal to the rim 29, wherein the contact sensor 51 is configured to detect when the rim 29 is in contact with the subject's body and sends a signal to the programmable chip 36. The contact sensor 51 upon determination of the establishment of a stable contact of the electronic stethoscope 10 with the subject's body, assesses if the pressure is less than the threshold value, the audio is attenuated facilitating prevention of any auditory spikes. Upon confirmation of contact between the diaphragm 23 and the subject's body, the programmable chip 36 initiates a timer, wherein the timer is remotely controlled according to an embodiment of the invention, and the remote-controlled timer enables a physician to define the recording window of the electronic stethoscope 10.

According to certain embodiments of the invention, the electronic stethoscope 10 discloses the contact sensor 51 positioned proximal to the rim 29 of the chest piece 24. The contact sensor 51 is configured to detect when the rim 29 establishes contact with the subject's body and to transmit a corresponding signal to the programmable processor or the programmable chip 36. Upon detection of a stable contact between the electronic stethoscope 10 and the subject's body, the contact sensor 51 further determines the applied pressure. If the detected pressure is below a predefined threshold value, the programmable chip 36 initiates attenuation of the audio signal to prevent auditory spikes or distortions caused by inadequate contact pressure. Conversely, if the pressure exceeds the threshold, the audio signal is processed normally without attenuation.

In one embodiment of the invention, upon confirmation of proper contact between the diaphragm 23 and the subject's body, the programmable chip 36 initiates a remote timer to define the duration of the auscultation or signal acquisition window. The remote timer may be remotely controlled, allowing a physician or other healthcare professional to define or adjust the recording duration of the electronic stethoscope 10 from a remote interface, facilitating enhancement of control, efficiency, and flexibility in both clinical and remote patient monitoring environments.

A variety of contact sensors may be employed for the purpose of monitoring contact between diaphragm 23 and the body. This allows the duration and quality of the measurement to be controlled. The contact sensor may be comprised of multiple elements. The contact sensor 51 may be a strain gauge or a force sensitive resistor (FSR), according to some embodiments of the invention. These elements, or combinations of these elements, and their respective electronic control chips combine to operate as contact sensors.

According to certain embodiments of the invention, the electronic stethoscope 10 is configured to process captured audio signals in real time, wherein the processing enables the generation and display of diagnostic information in both audio and visual formats. The electronic stethoscope 10 captures body sound signals via one or more sensors, and these signals undergo primary amplification, filtering, and anti-aliasing processing. The processed signal is then duplicated into two outputs. In the first output, the signal is routed to a microprocessor integrated with a Bluetooth module, enabling real-time playback of the body sounds for immediate auscultation. In the second output, the signal is transmitted to the main control module of the microprocessor, leading to generation of a real-time phonocardiogram display on an integrated liquid crystal display (LCD) screen. Additionally, the phonocardiogram data is transmitted to a software application hosted on an external electronic device such as a personal computer or portable terminal via a serial communication port or wireless communication protocols including Bluetooth or Wi-Fi.

Further, the LCD display screen of the external electronic device functions as a user interface or output unit for displaying the processed data received from the electronic stethoscope 10. The software application executing on the external electronic device is configured to display the phonocardiogram in real time after receiving and processing the body sound data through the main control module. The system may further include a data storage module for storing the acquired data and an auxiliary diagnostic module for performing computer-aided diagnosis, thereby enhancing the analytical and clinical utility of the device.

According to some embodiments of the invention, the electronic stethoscope 10 provides options for the end user to use either Bluetooth headset or an application on the electronic device of the user and data diagnosis in both real-time and offline modes. Further, the electronic stethoscope 10 is used in teleconsultation wherein the geolocation and the environmental conditions of the patient are captured during the diagnosis. In order to enhance the effectiveness of the remote healthcare services, the electronic stethoscope 10 is equipped to capture the end user's geolocation data and environmental conditions during the diagnostic process, facilitating diagnosis of the patient, where the healthcare provider is not physically present with the patient. Further, the distance measurements such as between the auscultation points or from anatomical landmarks is recorded. In addition, the geolocation data and environmental conditions of the patient such as location, altitude, temperature, humidity, background noise levels, and air quality etc., are also recorded through various sensors, as they affect the accuracy of the auscultation and are crucial to diagnose the patient.

Additionally, capturing the geolocation data and the environmental conditions along with the audio recording of the electronic stethoscope 10 ensures consistent and repeatable accurate placement of the electronic stethoscope 10. Further, it assists the remote healthcare providers in understanding the usage of the electronic stethoscope 10 during a teleconsultation session. Moreover, is also enables automated mapping of audio recordings to specific anatomical regions. The data facilitates diagnostics and interpretation of the symptoms. The combination of the geolocation data, the environmental conditions during the diagnostic process, combined with the patient data and auscultation audio, creates a comprehensive digital record that enhances the accuracy, repeatability, and documentation quality of remote or in-person diagnostic workflows, improves the accuracy of the remote assessments and enabling the personalized medical advice.

Accordingly, the electronic stethoscope 10 may also accommodate entry of individual profile information such as age, sex, ethnicity, etc. The display screen 33 and the control buttons 34 may be used to navigate questionnaires for simple answers. More complicated answers and background information may be captured via voice notes recorded from the patient or other onsite operator of electronic stethoscope 10. Display screen 33 may provide questions for more in-depth background information and voice notes associated with each question may be recorded.

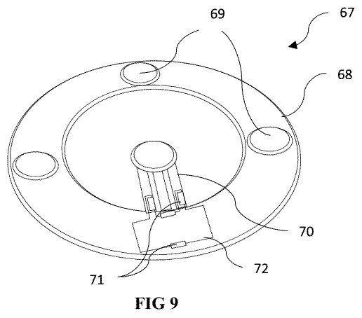

FIG. 9 is a perspective view of a tribosensor facilitating detection of ultra-sensitive audio, according to an embodiment of the invention. In the embodiment shown in FIG. 9, the sound sensor 41 in the electronic stethoscope 10 may be replaced with the tribosensor 67, wherein the tribosensor 67 comprises a diaphragm 68 for detecting or picking-up the body sounds, piezoelectric material 69 located on the diaphragm 68, a triple layer microcantilever 70, with piezoresistive material 71, connected by aluminum wires 72.

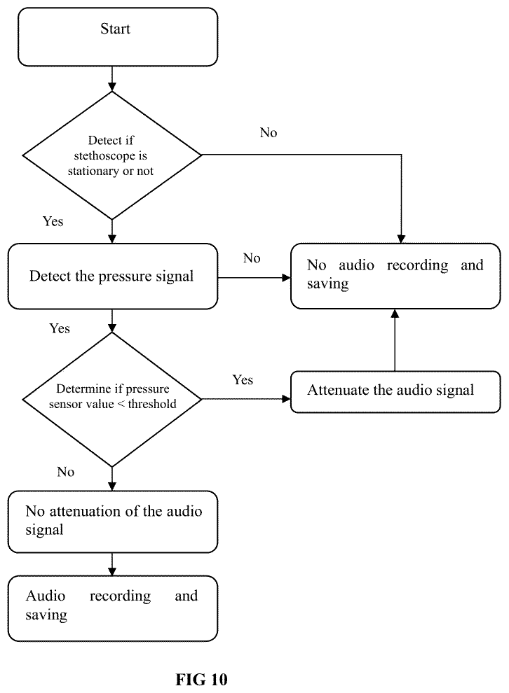

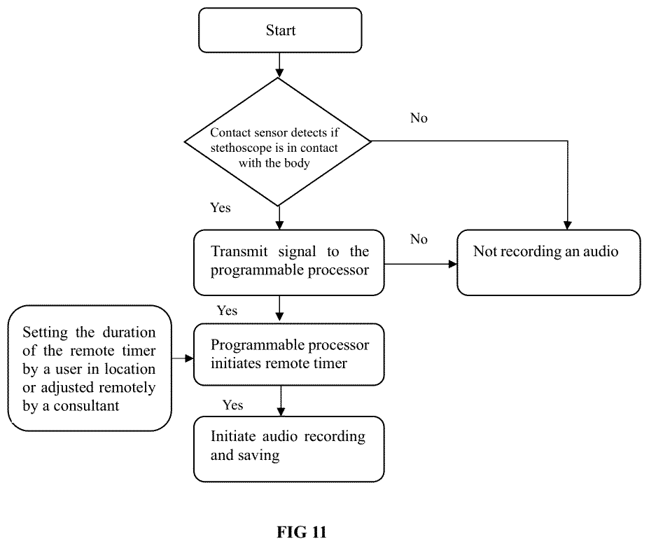

FIG. 10 illustrates a flowchart disclosing the working mechanism of the pressure signal to capture the audio recording. As disclosed in FIG. 10, the programmable chip 36 detects if the electronic stethoscope 10 is stationary or in movement. If the electronic stethoscope 10 is in stationary position, the pressure signal is detected by the contact sensor 51. Subsequently, if the electronic stethoscope 10 is not stationary, the audio recording is not performed. Furthermore, if the electronic stethoscope 10 is stationary but a pressure signal is not detected by the contact sensor 51, the audio recording is not performed. Upon detection of a pressure signal, the pressure value is evaluated, and if the detected pressure value is less than a predefined threshold, the corresponding audio signal is attenuated to suppress and the attenuated signals are not recorded or saved. Conversely, if the pressure value exceeds the threshold, indicating stable and sufficient contact with the subject's body, the audio signal is captured without attenuation. The resulting audio signal is then recorded and stored for subsequent analysis or transmission.

FIG. 11 illustrates a flowchart disclosing the working mechanism of the remote timer, to tune the remote timer. As disclosed in an embodiment in the FIG. 11, the contact sensor 51 positioned proximal to the rim 29 of the chest piece 24, wherein the contact sensor 51 is configured to detect when the rim 29 establishes contact with the subject's body and transmits a corresponding signal to a programmable processor 36. Upon confirmation of contact between the diaphragm 23 and the subject's body, the programmable processor 36 initiates the remote timer. The programmable processor 36 is further configured to operate a remote timer, that defines a time window for the audio recording based on the remote input received from a healthcare provider. The remote timer is communicatively coupled to the programmable processor 36, thereby enabling remote management of the duration of the audio recording, facilitating user-defined or physician-controlled control over the audio data acquisition interval from the sound sensor 41.

The electronic stethoscope 10 is capable of real-time analysis of a measurement, or processing a measurement to be stored, including remotely such as in cloud applications. When the electronic stethoscope 10 is supplying real time analysis, programmable chip 36 drives display 33 to show the appropriate information. For heart measurements, display 33 may show a graph of the heart beat and display the heart rate. For lung measurements, display 33 may show a frequency spectrum analysis of the lung sounds. According to some embodiments of the invention, a specific algorithm facilitates capturing the lung sounds by filtering out the body sounds, enabling isolation of the respiratory sounds for clearer analysis. The enhanced clarity in the captured lung sounds facilitates detection of the lung abnormalities, for example: detection of fine crackles, as seen in conditions such as fibrosis, detection of wheezes as seen in conditions such as asthma, Chronic Obstructive Pulmonary Disease (COPD) and the isolated respiratory sounds are used for artificial-intelligence based diagnostics or remote auscultation systems, according to some embodiments of the invention.

According to some embodiments of the invention, the sensor chip 43 is capable of applying different frequency filters to different body sound measurements. For example, a different filter may be applied to body sounds than is applied to lung sounds based on frequency window, wherein at least one of the microphones can be configured to gather the audio at 16 kHz sample rate. The received data is processed through filters having different cutoff frequencies. In one of the embodiments of the invention, the processing of the received cardiac sounds is achieved with low pass Finite impulse response (FIR) filter of variable weights and having cut-off frequency of 330 Hz in order to filter the high frequency noise, whereas the processing of lung sounds is achieved with the help of a high pass Infinite impulse Response (IIR) filter having the cut-off frequency of 100 Hz in order to filter the low frequency noise. Additionally, in another embodiment of the invention, filters such as bandpass filters and notch filters are used to filter the unwanted frequency audios from the intended signal. The input provided to the programmable chip 36 may result in sensor chip 43 applying different processing and filters to the signal from sound transducer 42. In the embodiment of central circuit board 30, the programmable chip 36 is also capable of wireless transmission and receiving of signals in at least Bluetooth and Wi-Fi modes, including in encrypted formats.

According to some embodiments of the invention, upon capturing a timed audio recording, the programmable chip 36 analyzes and compares the recording to an appropriate baseline selected from the sampled data. The baseline is selected using artificial intelligence programming in the programmable chip 36, according to an embodiment of the invention. The data and programming for analysis may be transmitted to the programmable chip 36 by a port or by wireless communication.

Moreover, the programmable chip 36 may do contrastive analysis between a current measurement and a baseline measurement, wherein the baseline measurement may be a previous measurement or set of previous measurements of a patient, or the baseline measurement may be developed from a database of measurements of a larger population. When, the comparison is to a database is performed, the artificial intelligence (AI) tools may be employed to select the appropriate baseline. In an initial step, an appropriate data set is chosen on a geographic basis. Differing geographic regions have different health profiles among the populations. These data sets are provided by health authorities, health counsels, etc. and consist of samples, or entries, for the individuals with each entry having multiple pieces of information pertaining to the respective individual of that entry. Among the pieces of information are at least one target piece of interest for the sake of comparison, such as a heart rate. Once a data set from the appropriate geographic region is selected, the bootstrapping and Random Forest techniques are applied to the data set.

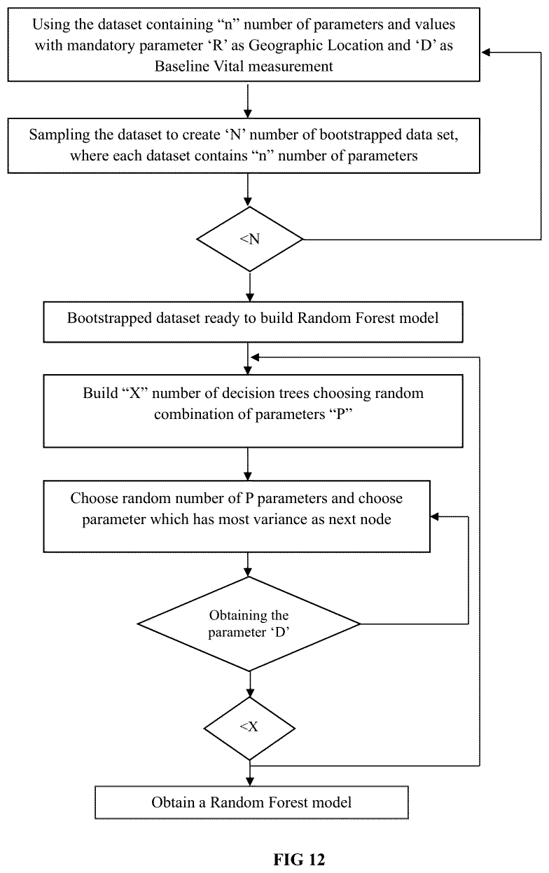

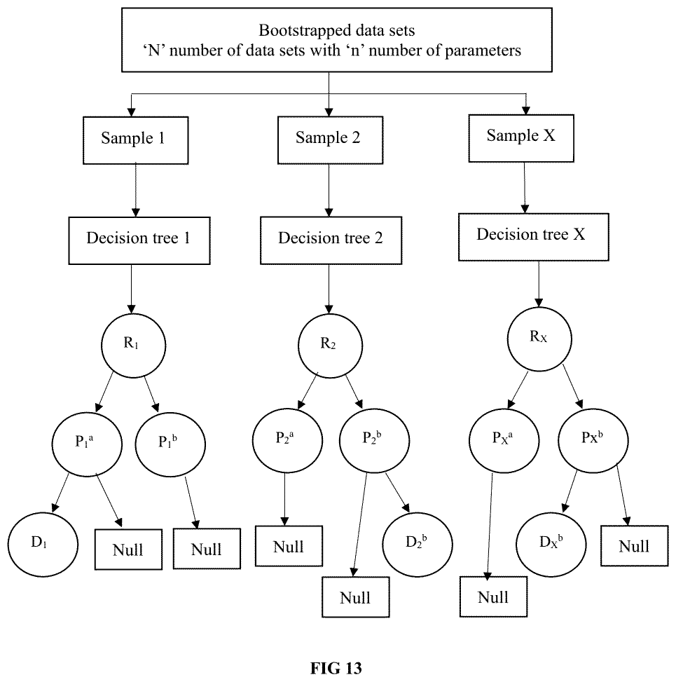

FIG. 12 and FIG. 13 illustrates the flowchart disclosing the process of building a random forest built with a bootstrapped data set, according to an embodiment of the invention. Bootstrapping is used to gather multiple samples of diagnostic measurements from huge dataset related to particular vital measurement from the same group of population, here from same geographic location and trained to form bootstrapped data set with higher accuracy. In an embodiment disclosed in the FIG. 12 and FIG. 13, the process of building the random forest model comprises the steps of using a data set containing “n” number of parameters and values with mandatory parameter R as geographic location and ‘D’ as baseline vital measurement and other parameters ‘P’. Further, the sampling is carried out on the data set to create “N” number of bootstrapped data sets, wherein each data set contains “n” number of parameters. The number of bootstrapped data sets collected for one vital would be ‘N’, where ‘N’ is more than 100,000. If the number of parameters in the bootstrapped data set less than “N′, the bootstrapped data set is ready to build a Random Forest model, and if the number of parameters is higher than ‘N’ then the step is repeated, until the number of parameters in the bootstrapped data set is less than ‘N’. In sampling the bootstrapped data, for each decision tree, a bootstrapped dataset is created by random sampling with replacement from the original dataset.

Further, the bootstrapped data set is used to build a random forest model, wherein as a first step, the random subset of the parameters ‘P’ from the bootstrapped dataset is used to build “X” number of decision trees to form a random forest. Additionally, the root node chosen is indicated as ‘R’. Further, an optimal number of subset parameters ‘P’ are chosen from the bootstrapped dataset, wherein from the optimal number of subset ‘P’, one subset is chosen that displays a huge variance in samples as root node ‘R’. According to an embodiment of the invention, the root node ‘R’ is the geographic region. Further, the next node of the decision tree from the remaining parameters is chosen by eliminating the root node parameter and choosing subset parameters ‘P’ from the bootstrapped dataset, wherein the one next node is chosen with which a tree is built with remaining parameters. This process is repeated until the decision parameter ‘D’ or the vital measurement is obtained. Additionally, the above process is repeated to form multiple decision trees that builds a random forest model. The process is repeated ‘X’ number of times to form a variety of decision trees facilitating building of the random forest model, the obtained random forest model is trained by using the patients' data. According to some embodiments of the invention, the decision trees are built for each bootstrapped data set, wherein at each node, the root node ‘R’ is randomly selected, where ‘R’ is the geographic location. The data is split at each node based on the best feature from the random subset chosen from other parameters, and the decision tree is built until last parameter is ‘D’, the vital measurement to infer.

By combining the method of bootstrapping and the random forest algorithm, the diagnosis of the patient using the patient's vitals is inferred, where according to an embodiment of the invention, the heart rate is a vital that is being diagnosed. The method is not limited to heart rate and is also valid to diagnose other vital measurements such as heart systolic, heart diastolic, myocarditis, pericarditis, Pneumonia etc.,

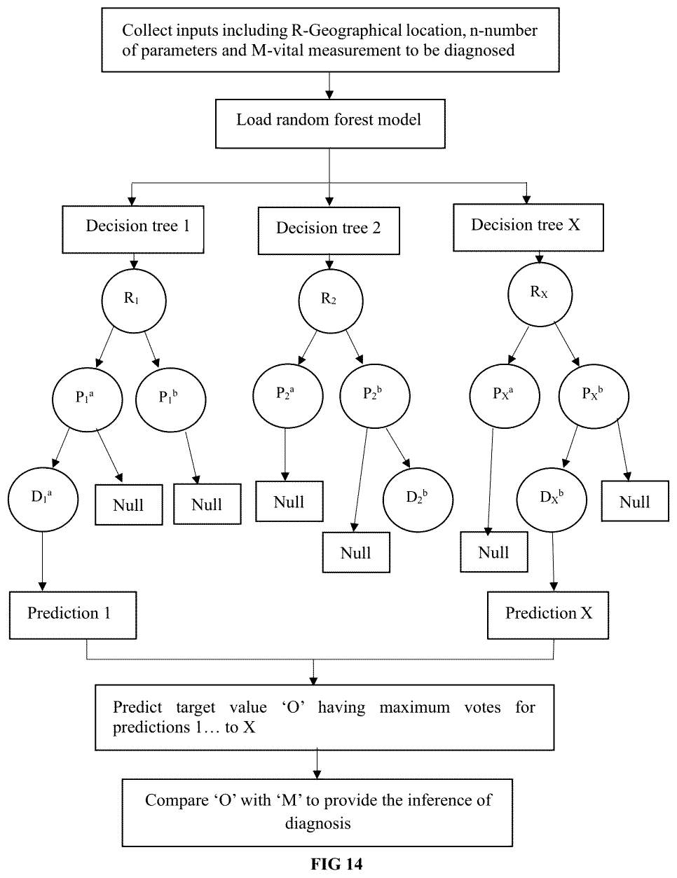

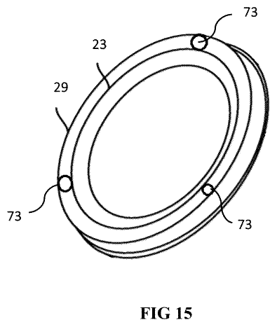

FIG. 14 illustrates the flowchart disclosing the diagnostic inference using the random forest built with the bootstrapped data set, according to an embodiment of the invention. In the embodiment disclosed in FIG. 14, the patients' data is the collected, wherein “n” number of parameters related to the patient are collected, where “M” is the vital measurement that is to be diagnosed. The random forest model is loaded and “n” numbers of parameters are provided to the random forest model respectively, wherein the patients' data in the input parameters “n” is processed through each of the decision tree in the random forest, and the target vital measurement is measured. The random forest model processes the input data and predicts the target vital value “O” parameter for diagnosis. According to an embodiment of the invention, the target vital value “O” is determined by considering the parameter that received most votes that is close to the diagnosis. The data collected from the patient that has parameters “n”, wherein the “n” parameters are same as the parameters of the bootstrapped data set related to the vital measurement and the electronic stethoscope 10 measures the vital measurement “M”, wherein “O” and “M” are compared to derive at the diagnosis. The processer in the electronic stethoscope 10 compares the vital measurement output “O” from random forest with the patient's vital measured by the electronic stethoscope 10 and provides diagnosis report. For example, if the vital measurement is for the heart rate, the random forest model measures if heart rate if normal, high or low.

The input data is provided to the random forest model, wherein the random forest model processes the input data and the output obtained by processing the data through the random forest model is the target or the healthy vital measurement of the patient based on the parameters collected (i.e., based on the demographics/age/sex/weight etc. of the patient).

According to an embodiment of the invention, the selected data set is an accurate representation of the relevant population and bootstrapping is used to develop additional data sets from the data set of actual samples, or entries. A size is selected for the bootstrapped data sets to be equal to or less than the sample data set size. The samples, or entries, are randomly selected from the original sample data set and added to the bootstrapped data set in process until the bootstrapped data set has the desired number of entries, wherein the samples are chosen using a random number generator. Once the bootstrapped data set has the correct number of copied samples, the data set is considered complete and is ready for the analysis or processing. Further, other bootstrapped data sets are constructed from the original sample data set. Additionally, after multiple bootstrapped data sets are constructed from the original sample data set, the Random Forest algorithm is applied to the datasets. The bootstrapped data and the Random Forest algorithms are applied to determine the target value for a health parameter.

Additionally, according to an embodiment of the invention, the electronic stethoscope 10 uses adaptive spectral subtraction to identify the regions of constant (clipped) amplitude and replaces these regions using cubic spline interpolation. When the microphone reached the maximum acoustic input, a clipping distortion algorithm is applied to correct for the truncated signal amplitude. The electronic stethoscope 10 employs the adaptive spectral subtraction algorithm to enhance the quality of the recorded auscultation signal, wherein the use of adaptive spectral subtraction is particularly effective in identifying and mitigating regions where the amplitude of the recorded signal has been clipped due to the microphone reaching its maximum acoustic input threshold. The clipping distortion occurs when the input signal exceeds the dynamic range of the microphone, resulting in a truncated waveform that loses its original shape and introduces significant harmonic artifacts and distortions.

Although these clipped segments are typically limited to just a few samples per instance, the clipped regions produced prominent signal distortions such as non-linear distortions that significantly degrade the quality and diagnostic reliability of the audio signal. Hence, the present invention uses adaptive spectral subtraction to isolate the clipped regions, that involves analyzing the spectral content of the audio signal and dynamically estimating the noise floor or distortion spectrum introduced by clipping mechanism. By subtracting the estimated distortion spectrum, the algorithm preserves and enhances the quality of the undistorted portions of the signal. Upon identification, the clipped regions are reconstructed using cubic spline interpolation, a mathematical technique that generates a smooth curve through known data points. The cubic spline method ensures that the reconstructed segments seamlessly integrate with the surrounding waveform, preserving both the spectral and temporal characteristics of the original signal. The combined approach of using the adaptive spectral subtraction and the cubic spline interpolation enables the electronic stethoscope to correct the clipped audio artifacts in real-time or during post-processing, thereby delivering clear, more accurate heart and lung sounds for diagnostic interpretation. It is especially valuable in high-volume or noisy environments where loud body sounds or improper sensor placement may trigger microphone saturation.

Further, the electronic stethoscope 10 comprises the sound aperture located in the handle of the fixed housing 20, where the sound transducer 42 facilitates recording an external sound moving through the aperture or a voice note. The programmable processor 36 is equipped with a voice note capture module that enables users, such as clinicians or healthcare professionals to attach the verbal annotations to each patient examination or audio recording. The programmable processor or programmable chip recognizes the simple voice commands to navigate and record the voice notes. The voice note feature is particularly valuable for recording diagnostic impressions, timestamped observations, or contextual information in real time, without interrupting the workflow. The voice note capture module facilitates the voice annotation, improving the documentation quality, reducing manual input during high-pressure scenarios, enabling faster post-examination review or integration into the patient records etc. The voice data is stored alongside the corresponding auscultation recording, either locally or wirelessly transmitted to a companion device or cloud storage, depending on system configuration. Furthermore, the complicated answers from the patients and their background information may be captured via voice notes recorded from the patient or other onsite operator of electronic stethoscope 10. Display screen 33 may provide questions for more in-depth background information and voice notes associated with each question may be recorded.

Further, in some embodiments of the acoustic electronic stethoscope 10 as disclosed in FIG. 15, comprises at least three electrodes 73 facilitating measurement of body sounds of the patient, wherein the electrodes 73 are adapted to contact the skin of the patient and measure the electrical signals from the body of the patient, wherein the wireless communication device is adapted to transmit the Electrocardiogram (ECG) waveform and process the Electrocardiogram (ECG) waveform for the additional information related to the Electrocardiogram (ECG). Further, the electrodes 73 include metal contacts made of silver, and further contains the disposable pad such as tabs and are located on extendable arms, according to an embodiment of the invention.

Additionally, the electronic microscope 10 comprises power saving mechanism such as sleep time implementation on the sensors such as pressure sensor or capacitive touch sensor, according to an embodiment of the invention, wherein implementing a sleep time for the sensors reduces the power consumption when the electronic stethoscope 10 is not in use. The reduction in power consumption is achieved by maintaining the electronic stethoscope 10 in a low-power or sleep mode during periods of inactivity, and transitioning the device to an active operational state upon detection of pressure or touch input. In some embodiments, a pressure sensor is employed to facilitate activation of the electronic stethoscope 10. Upon detection of the pressure by the pressure sensor, the signal is transmitted to the microcontroller, which in turn activates the electronic stethoscope 10, rendering it ready to capture the audio. This configuration enables efficient power management while ensuring immediate responsiveness when user interaction is detected. Subsequently, in case of no activity for a preset duration of time, for example: 5 minutes, the electronic stethoscope 10 returns to sleep mode.

According to some embodiments of the invention, the capacitive touch sensor is also used with the electronic stethoscope 10, wherein the electronic stethoscope 10 gets activated upon coming in contact with a user or when a user touches the stethoscope. In sleep mode, the components of the electronic stethoscope 10 remain in sleep mode whereas the capacitive touch sensor is always activated, monitoring the touch. When touch is detected, a triggering mechanism activates the microcontroller, and the electronic stethoscope 10 is activated and is ready to use. In case where touch is not detected, the electronic stethoscope 10 remains in sleep mode. According to some embodiments of the invention, the accelerometer and the capacitive touch sensor are used in combination, thus enabling motion detection and touch detection for smarter wake-up mechanism.

We claim:

1. An electronic stethoscope for recording sounds produced from within a body, the electronic stethoscope comprising:
a fixed housing defining an integrated head and a handle for manual control, wherein the integrated head and handle provide for single hand for manual movement;
a static diaphragm at an exterior surface of the integrated head of the fixed housing and configured to be retained by a rim, wherein the static diaphragm further comprises a sensor module configured to detect a body sound;
a chest piece behind the diaphragm within the fixed housing, wherein the chest piece is replaceable and is secured in a chest-piece holder, where the chest piece has an aperture through it;
a sound sensor located outside of and behind the chest piece within the fixed housing, where the sound sensor is positioned to receive sound from the aperture in the chest piece, where the aperture in the chest piece facilitates placement of the sound sensor at the end of an audio tube, where the audio tube connecting the sound sensor and the chest piece, configured to conduct the body sounds;
an ambience microphone located within the fixed housing configured to record ambient sound;
an anti-chill ring, located at the outer side of the chest piece, configured to prevent the direct contact of the chest piece with the body;
a visible-cue LED indicator configured to indicate an ON or OFF state of the electronic stethoscope, wherein the visible-cue LED indicator is further configured to provide a visual indication when the electronic stethoscope is ready to capture audio data, and wherein the visible-cue LED indicator is further configured to provide a visual indication, upon completion of audio capture;
a contact sensor disposed on the rim, configured to sense contact pressure and determine the establishment of stable contact of the electronic stethoscope with the body, and further configured to determine whether the electronic stethoscope remains stationary while in contact with the body;
a programmable processor configured to attenuate an audio signal when a pressure value detected by the contact sensor is below a predefined threshold, and to enable capturing of the audio signal when the pressure value exceeds the predefined threshold;
wherein the programmable processor is configured to monitor the contact sensor for a contact signal indicating the diaphragm being positioned against the body upon manual control of a handle;
wherein the programmable processor is further configured to operate a remote timer communicatively coupled to the programmable processor, the remote timer being configured to define a time window for audio recording based at least in part on a remote input;
wherein the duration of the audio recording is determined based on the contact signal generated by the contact sensor while in contact with the body, and the remote input provided to the remote timer.

2. The electronic stethoscope as claimed in claim 1 wherein, the static diaphragm further comprising a sensor module, where the static diaphragm and the sensor module are configured to measure the vibrations from the body and to prevent air entering the chest-piece, enabling measurement of the auscultation signal.

3. The electronic stethoscope as claimed in claim 1 wherein, the sensor module in the static diaphragm further comprises a one directional sensor, enabling noise cancellation to facilitate capturing body sounds.

4. The electronic stethoscope as claimed in claim 1 wherein, the remote timer configured to record and control the time of measurement, whereby the remote timer is further configured to define a recording window of the electronic stethoscope.

5. The electronic stethoscope as claimed in claim 1 wherein, the contact sensor is configured to terminate audio recording when the duration of contact between the electronic stethoscope and the subject exceeds a threshold duration determined at least in part by the contact sensor; wherein the electronic stethoscope is further configured to enable audio capture for a predetermined duration defined by a remote timer, irrespective of continued contact with the body.

6. The electronic stethoscope as claimed in claim 1 wherein, the visible-cue LED indicator is configured to indicate an operational status of the electronic stethoscope, wherein the LED indicator emits a first signal to indicate that a measurement is in progress, and emits a second signal to indicate that the measurement has been successfully completed.

7. The electronic stethoscope as claimed in claim 1 wherein, the programmable processor is further configured to:
a. attenuate the audio signal to avoid auditory spikes, when the detected pressure is below the predetermined threshold upon making contact, excluding to record the audio;
b. suppress the auditory spike by attenuating the audio signal during processing, or record the audio signal without attenuation when the pressure detected by the contact sensor is above a predetermined threshold, facilitating creation of a readable file.

8. The electronic stethoscope as claimed in claim 1 wherein, the programmable processor is further configured to:
invert the ambient noise signal received from the ambience microphone and combine the inverted ambient noise signal with the audio signal received from the sound transducer, to exclude at least a portion of the ambient noise component present in the combined audio signal, resulting in an output signal primarily representing the body sounds detected by the sound transducer.

9. The electronic stethoscope as claimed in claim 1, wherein the programmable processor is configured to:
apply a frequency filter to different body sound measurements;
transmit and receive a wireless signal and produce the audible sound to be emitted from a sound generator in response to one of a beginning of recording, an ending of recordings, or an error condition;
wherein, the captured audio is transmitted to the programmable processor, the programmable processor further configured to generate audio and visual outputs based on the captured audio signal, thereby facilitating real-time diagnosis.

10. The electronic stethoscope as claimed in claim 1 wherein, the electronic stethoscope is configured to capture a recording of body sounds;
wherein, the programmable processor or programmable chip of the electronic stethoscope is configured to compare one or more metrics derived from the audio recording to one or more baselines;
wherein the baseline is selected by:
(a) applying a bootstrap algorithm to a sampled dataset to generate one or more additional datasets; and
(b) applying a random forest algorithm to one or more additional datasets to determine the baseline for each data set;
wherein at least one baseline is selected based on geographic data; and
wherein at least one baseline is selected based on one or more previous measurement, a set of previous measurements, or
measurements derived from a larger population.

11. The electronic stethoscope as claimed in claim 10 wherein, the electronic stethoscope is configured to obtain one or more vitals of the subject, and wherein a diagnostic system employs a random forest model trained using a bootstrapped data set, the model configured to process the obtained vitals to facilitate determination of a diagnosis for the patient.

12. The electronic stethoscope as claimed in claim 10 wherein, the stethoscope is configured to gather a body sound obtained by the sound sensor, and further configured to process the signal through amplification, filtering, anti-aliasing to generate a first output and a second output;
wherein the first output is transmitted to a microprocessor of a Bluetooth module for real-time playback of the body sound;
wherein the second output is transmitted to a microprocessor of a main control module located on an electronic device of a user for displaying a real-time phonocardiogram on an LCD display screen;
wherein the second output is further transmitted to an application executing on the electronic device via a serial port or a communication module.

13. The electronic stethoscope as claimed in claim 10 wherein, the main control module in the electronic device of the user configured to process the body sound signal;
wherein, the main control module further configured to display the phonocardiogram in real time on the display screen of the electronic device;
wherein, a storage module in the electronic device of the user is configured to store the data;
wherein, an auxiliary diagnosis module in the electronic device of the user configured to perform computer-aided diagnosis.

14. The electronic stethoscope as claimed in claim 10 wherein, further comprising the aperture defined in the handle of the fixed housing;
a sound transducer configured to record one or more external sounds moving through the sound apertures in the fixed housing, wherein the one or more external sound is a voice note;
wherein the programmable processor is further configured to employ a voice note module to recognize at least one voice command for navigating and recording the voice note.

15. The electronic stethoscope as claimed in claim 10 wherein, the programmable processor is configured to perform adaptive spectral subtraction to identify one or more regions of constant clipped amplitude in a captured audio signal;
wherein the programmable processor is further configured to replace the identified regions using cubic spline interpolation;
wherein the programmable processor is further configured to apply a clipping distortion algorithm to adjust the truncated signal amplitude when the ambient microphone reaches a maximum acoustic output level.

16. The electronic stethoscope as claimed in claim 10 wherein, the programmable processor is configured to monitor the contact sensor for the contact signal indicating that the diaphragm is in stationary position;
the programmable processor further configured to operate a remote timer to control the duration of a recording from the sound sensor;
wherein, the contact sensor comprises at least one of a force-sensitive resistor (FSR), strain gauge, mechanical switch, or capacitive proximity sensor.

17. The electronic stethoscope as claimed in claim 10 wherein, the programmable processor is configured to continue recording the audio signal from the sound sensor only while the contact sensor transmits a contact signal.

18. The electronic stethoscope as claimed in claim 10 wherein, the contact sensor is configured to sense the force of contact, wherein the programmable processor requires a minimum force to maintain a minimum threshold for the duration of the recording.

19. The electronic stethoscope as claimed in claim 10 wherein, the programmable processor is configured to analyze the signal from the contact sensor to determine whether the diaphragm is in stationary position on the body and is further configured to record the signal from the sound sensor only when the diaphragm is in stationary position.

20. The electronic stethoscope as claimed in claim 13 wherein, the electronic stethoscope is in communication with external elements, where the external elements comprise at least one of the following;
a) cloud servers, or
b) a Bluetooth client.

21. The electronic stethoscope as claimed in claim 10 wherein, the Bluetooth of the electronic stethoscope is configured to be continuously active and ready to capture the audio recording.

22. The electronic stethoscope as claimed in claim 1 wherein, the electronic stethoscope further comprising a tribosensor integrated on the sound sensor facilitating detection of ultra-sensitive audio.

23. The electronic stethoscope as claimed in claim 1 wherein, the electronic stethoscope further comprises a smart ambient noise detection feature configured to automatically adjust a noise-cancellation level based on the detected ambient noise where the electronic stethoscope is being used,
wherein the conditions of the ambient noise include at least a loud setting and a quiet setting;
and wherein the detected ambient noise level along with an intelligent noise-adjustment mechanism is applied to the audio signal received from the sound sensor.
